(19) United States
(12) Patent Application Publication
(10) Pub. No.: US 2005/0119517 A1
(43) Pub. Date: Jun. 2, 2005

(54) Process for upgrading a liquid hydrocarbon stream

(76) Inventors: Christopher Russell Millington, Chester (GB); Arian Nijmeijer, Amsterdam (NL)

Correspondence Address: Yukiko Iwata;c/o Shell Oil Company, Intellectual Property, P.O. Box 2463, Houston, TX, 77252-2463, US

(21) Appl. No.: 10/981,422

(22) Filed: Nov. 4, 2004

(30) Foreign Application Priority Data
Nov. 4, 2003 (EP) 03256952.7

(51) Int. Cl. C07C 7/144; C10G 31/11

(52) U.S. Cl. 585/818; 208/177

(57) ABSTRACT

A process for upgrading a liquid hydrocarbon transportation fuel is provided, wherein an inlet stream of liquid hydrocarbon transportation fuel, preferably diesel or gasoline base fuel is contacted with a non-porous or nano-filtration membrane and a first liquid hydrocarbon outlet stream is recovered as the retentate and a second liquid hydrocarbon outlet stream is recovered as the permeate. The retentate is more than 70 weight % of the inlet stream. The inlet stream and the first and the second outlet stream each fulfil the requirements for base fuel without further treatment.

### FIELD OF THE INVENTION

The invention relates to a process for upgrading a liquid hydrocarbon stream, and, more particularly, for upgrading a liquid hydrocarbon transportation fuel.

### BACKGROUND OF THE INVENTION

Contaminants such as polynuclear aromatics, organometal compounds, water, and salt can be removed from liquid hydrocarbon streams such as gasoline, gasoil, naphtha and kerosene by means of membrane separation.

In U.S. Pat. No. 5,133,851 for example is described a process for the reduction of the metal content of a hydrocarbon feed mixture consisting essentially of kerosene or gasoil. The kerosene or gasoil is contacted with a metal-selective membrane. Polydimethylsiloxane is mentioned as a particularly preferred membrane material. As permeate a product is obtained that comprises at least about 70% wt of the hydrocarbon feed mixture. A hydrocarbon retentate fraction having a greatly enhanced metal content is obtained.

In U.S. Pat. No. 5,962,763 the removal of hydrocarbons with a high boiling point (above 480° C.) and/or salt from a stream of light hydrocarbons such as naphtha and gasoil is described. The contaminated stream of light hydrocarbons is supplied to a membrane and separated into a permeate stream and a small retentate stream. Polydimethylsiloxane is mentioned as a suitable material for a membrane to separate hydrocarbons with a high boiling point from a hydrocarbon stream.

In WO 01/60949, a process is described for purifying transportation fuels comprising at most 5 wt % of high molecular contaminants by contacting the fuel with a hydrophobic non-porous or nanofiltration membrane. The membrane is preferably a cross-linked polysiloxane membrane. The stage cut—defined as the weight percentage of the original fuel that passes through the membrane and is recovered as permeate—may vary from 30 to 99% by weight, preferably 50 to 95% by weight.

In the aforementioned processes, the retentate stream is relatively small and thus contains a relatively high amount of contaminants. This implies that the retentate stream has to be cleaned or further processed before it can be used as a commercial product. Especially at depots or retail sites for transportation fuels, cleaning or further processing facilities are not generally available.

U.S. 2003/0173255 (White et al.) describes a selective membrane separation process in which a hydrocarbon-containing naphtha feed stream is contacted with a membrane separation zone containing a membrane having a sufficient flux and selectivity to separate a permeate fraction enriched in aromatic and monoaromatic hydrocarbon containing sulphur species, and a sulphur-deficient retentate fraction. The sulphur-deficient retentate comprises no less than 50% by weight of the feed, and preferably contains at least 70% by weight, preferably at least 80% by weight of the total feed passed over the membrane (paragraph [0026]). Typically (paragraph [0016]), the hydrocarbon streams contain greater than 150 ppmw, preferably from about 150 ppmw to about 3000 ppmw, most preferably from about 300 ppmw to about 1000 ppmw, sulphur. The (sulphur-enriched) permeate fraction is subjected to a (further) non-membrane process to reduce sulphur content. This non-membrane process is a conventional sulphur removal technology, e.g. hydrotreating (paragraph [0012]).

The point of the process of U.S. 2003/0173255 is to reduce amount of hydrocarbon requiring hydrotreatment, both for reasons of costs and to avoid hydrogenation of olefin and naphthene compounds in fluid catalytic cracking (FCC) naphtha (paragraphs [0004], [0012]).

U.S. 2002/0007587 (Geus et al.) describes a process for purifying a liquid hydrocarbon fuel comprising 5% by weight or less of high molecular weight contaminants, which process comprises contacting the fuel with a hydrophobic non-porous or nano-filtration membrane to produce a purified product stream, and recovering the purified product stream as permeate (paragraph [0008]). The weight percentage of permeate as a percentage of feed can vary within broad limits: 30 to 99% by weight, preferably 50 to 95% by weight (paragraph [0010]). In the examples, the permeate constitutes 66% by weight of the gasoline feed. There is no disclosure relating to the retentate, which, however, could be purified by distillation as per paragraph [0005].

WO-A-01060771 discloses a process for purifying a liquid hydrocarbon product comprising 5% by weight or less of high molecular weight contaminants having a molecular weight of at least 1000, wherein the product stream is contacted with a hydrophobic non-porous or nano-filtration membrane and the purified product stream is recovered as the permeate. Typically, the liquid hydrocarbon product is a polymerisable hydrocarbon such as dicyclopentadiene, and the process steam that passes through the membrane and is recovered as permeate can vary within broad limits: 10 to 99% by weight, preferably 30 to 95% by weight.

Although there is no specific limitation as to the nature of the liquid hydrocarbon product in WO-A-01060771, the products specifically mentioned are all industrially produced chemical product streams, particularly those containing a polymerisable olefinic bond. The products may include one or more heteroatoms, and named examples of liquid hydrocarbon products include hydrocarbon per se, such as cyclopentadiene, dicyclopentadiene, 1,3-cyclohexadiene, cyclohexene, styrene, isoprene, butadiene, cis-1,3pentadiene, trans-1,3-pentadiene, benzene, toluene, xylenes, ethene and propene. Named liquid hyrocarbon products containing heteroatoms are methyl acrylate, ethyl acrylate and methylmethacrylate. There is no mention in WO-A-01060771 of liquid hydrocarbon transportation fuel.

### SUMMARY OF THE INVENTION

Accordingly, a process for upgrading a liquid hydrocarbon transportation fuel is provided comprising contacting an inlet stream of liquid hydrocarbon transportation fuel with a non-porous or nano-filtration membrane to produce a first liquid hydrocarbon outlet stream recovered as the retentate and a second liquid hydrocarbon outlet stream recovered as the permeate, wherein the retentate is more than 70 weight % of the inlet stream, and wherein the inlet stream and the first and the second outlet stream each fulfill the requirements for base fuel without further treatment.

### DETAILED DESCRIPTION OF THE INVENTION

It has now been found that it is possible to use a non-porous or nano-filtration membrane to separate a liquid hydrocarbon transportation fuel into a permeate and a retentate in such a way that no cleaning or further processing of the retentate is needed. The retentate can be used for the same purpose as the inlet hydrocarbon stream without additional cleaning or processing. As permeate a high quality product is obtained—for example a choice grade transportation fuel that can be sold as a premium product.

Accordingly, an inlet stream of liquid hydrocarbon transportation fuel is contacted with a non-porous or nano-filtration membrane and a first liquid hydrocarbon outlet stream is recovered as the retentate and a second liquid hydrocarbon outlet stream is recovered as the permeate. The retentate is more than 70 weight % of the inlet stream, and the inlet stream and the first and the second outlet stream each fulfil the requirements for base fuel without further treatment.

In the process according to the invention, an inlet stream of liquid hydrocarbon transportation fuel is led over a non-porous or nanoporous membrane that is resistant to hydrocarbons. A first outlet stream of liquid hydrocarbons is recovered as the retentate and a second outlet stream of liquid hydrocarbons is recovered as the permeate. The process conditions in the process according to the invention are chosen such that more than 70 weight % of the inlet stream is withheld by the membrane as retentate.

In the process according to the invention, an inlet stream that can be used for transportation fuel is separated into a retentate stream that can still be used for that same purpose, since it still fulfils the quality and composition requirements, and a high quality permeate stream. An advantage of the process is thus that a high quality permeate stream, i.e. having a higher quality than the inlet stream, can be produced whilst obtaining a retentate stream that has substantially the same quality as the inlet stream.

The liquid hydrocarbon stream may for example be a transportation fuel such as kerosene, diesel or gasoline. Preferably, the liquid hydrocarbon stream is a diesel or (most preferably) gasoline base fuel.

Reference herein to a diesel or gasoline base fuel is to a hydrocarbon stream boiling in the diesel or gasoline boiling range, that is without further treatment suitable as a commercial grade diesel or gasoline base fuel. Additives might be added to the base fuel before they are used in an internal combustion engine. Those skilled in the art will appreciate that addition of additives does not constitute “further treatment” of the base fuel. Gasoline and diesel additives are known in the art and include, but are not limited to, anti-oxidants, corrosion inhibitors, detergents, dehazers, dyes and synthetic or mineral oil carrier fluids.

Gasoline base fuels typically contain mixtures of hydrocarbons boiling in the range from about 30° C. to about 230° C., the optimal ranges and distillation curves varying according to climate and season of the year. Diesel base fuels typically contain mixtures of hydrocarbons boiling in the range from about 150° C. to about 400° C.

It is an advantage of the process according to the invention that no waste stream or contaminated stream that has to be cleaned or further processed is produced. All liquid hydrocarbon streams produced are commercial grade hydrocarbon streams. This makes the process according to the invention particularly suitable to be applied at fuel depots or at retail sites, where no or limited processing facilities are available.

It will be appreciated that the stagecut, i.e. the weight % of the inlet stream that permeates through the membrane, will be chosen such that the retentate, i.e. the first outlet stream, can still be used without further treatment (further processing). The exact stage-cut therefore depends inter alia on the composition and quality of the inlet stream. The inlet stream preferably contains less than 150 ppmw (parts per million by weight) sulphur, more preferably less than 140 ppmw (e.g. 138 ppmw) sulphur, and advantageously less than 50 ppmw sulphur (e.g. less than 25 ppmw sulphur, for example 22 ppmw sulphur).

Preferably, at least 80 weight % of the inlet stream is withheld by the membrane as retentate, more preferably the retentate is 85 to 95 weight % of the inlet stream. The desired stage cut can be set by setting the flow and/or trans-membrane pressure for a given permeability of the membrane.

The process according to the invention can advantageously be applied at a gasoline or diesel depot to produce choice grade gasoline or diesel base fuel (permeate) from the main grade base fuel that is stored at that depot. The retentate that is obtained is also a main grade gasoline or diesel base fuel, although it might differ in some quality aspects from the inlet base fuel. In order to avoid the need for two different storage tanks for the two different main grade base fuels (inlet and retentate) at such depot, it is preferred that the main grade base fuel that is produced as retentate is directly loaded into a transport truck. It is an advantage of the process according to the invention that start-up and shut-down is very easy, since a membrane unit can easily be switched on or off. Thus, in case of direct truck loading, the process will only be carried out if and when a transport truck is available for loading of the main grade base fuel.

Suitable membranes for the process according to the invention are non-porous or nanoporous membranes that are resistant to hydrocarbons. Suitable nanoporous membranes are for example ceramic membranes or nanoporous polymeric membranes. These membranes are known in the art. Examples of nanoporous polymeric membranes are cellulose acetate, modified cellulose, polyamide, polyimide, polyetherimide, polyaramide and polyethersulphones.

Preferably, the membrane is a hydrophobic non-porous membrane. The hydrophobic non-porous membrane is typically supported on at least one porous substrate layer to provide the necessary mechanical strength. The combination of non-porous membrane and porous substrate layer is often referred to as composite membranes or thin film composites. The non-porous membrane may also be used without a substrate, but it will be understood that in such a case the thickness of the membrane should be sufficient to withstand the pressures applied. A thickness greater than 10 μm may then be required. This is not preferred from a process economics viewpoint, as such thick membrane will significantly limit the throughput of the membrane. The membrane may have a thickness of from 0.5 μm, preferably of from 1 μm, to 30 μm, to preferably 10 μm.

In case a non-porous membrane is used, transmission of the permeate takes place via the solution-diffusion mechanism: the hydrocarbons to be permeated dissolve in the membrane matrix and diffuse through the thin selective membrane layer, after which they desorb at the permeate side. The main driving force for permeation is hydrostatic pressure.

Hydrophobic, non-porous membranes as such are known in the art and in principle any hydrophobic non-porous membrane through which gasoline can be transmitted via the solution-diffusion mechanism, can be used. Typically such membranes are cross-linked to provide the necessary network for avoiding dissolution of the membrane once being in contact with a liquid hydrocarbon product. Cross-linked non-porous membranes are well known in the art. In general, cross-linking can be effected in several ways, for instance by reaction with cross-linking agents, and can optionally be enhanced by irradiation.

Examples of suitable, presently available cross-linked non-porous membranes are cross-linked silicone rubber-based membranes, of which the cross-linked polysiloxane membranes are a particularly useful group of membranes. Cross-linked polysiloxane membranes known in the art can be used, for example from U.S. Pat. No. 5,102,551. Typically, the polysiloxanes contain the repeating unit —Si—O—, wherein the silicon atoms bear hydrogen or a hydrocarbon group. Preferably the repeating units are of the formula (I)
—[Si(R)(R′)—O—]ₙ— (I)

In the above formula, R and R′ may be the same or different and represent hydrogen or a hydrocarbon group selected from the group consisting of alkyl, aralkyl, cycloalkyl, aryl, and alkaryl. Preferably, at least one of the groups R and R′ is an alkyl group, and most preferably both groups are alkyl groups. Very suitable cross-linked polysiloxane membranes for the purpose of the present invention are cross-linked polydimethylsiloxane membranes or cross-linked polyoctylmethylsiloxane membranes. Preferred polysiloxane membranes are cross-linked elastomeric polysiloxane membranes.

Also other rubbery non-porous membranes could be used. In general, rubbery membranes can be defined as membranes having a non-porous top layer of one polymer or a combination of polymers, of which at least one polymer has a glass transition temperature well below the operating temperature, i.e. the temperature at which the actual separation takes place. Yet another group of potentially suitable non-porous membranes are the so called superglassy polymers. An example of such a material is polytrimethylsilylpropyne.

As indicated hereinbefore the non-porous membrane may be used as such, but is preferably supported on a substrate layer of another material. Such substrate layer could be a macroporous or mesoporous substrate layer. Examples of suitable substrate materials are polyacrylonitrile (PAN), polyether imide (PEI) or poly imide (PI).

Various types of membrane units may be applied in the process according to the invention, such as flat sheet, spiral wound or hollow fibre membrane units, preferably a flat sheet or spiral wound membrane unit.

It is preferred that the inlet stream is contacted with the membrane at a trans-membrane pressure in the range of from about 2 to about 80 bar, more preferably from about 10 to about 50 bar. The flux is typically in the range of from about 200 to about 5000 kg per square metre membrane per day (kg/m²d), preferably at least 250 kg/m²d.

It will be appreciated that the operating temperature depends inter alia on the membrane material that is used. For polymeric membranes, the temperature is preferably in the range of from about 10° C. to about 80° C., more preferably from about 10° C. to about 40° C. For ceramic membranes, the operating temperature may be higher, but will be limited by the boiling point of the inlet stream. For gasoline for example, the operating temperature will be below 100° C. in order to have a liquid inlet stream.

### EXAMPLES

The invention will be illustrated by means of the following non-limiting examples, in which temperatures are in degrees Celsius and, unless otherwise indicated, parts and percentages are by weight.

### Example 1

A gasoline inlet stream (composition and properties as shown in Table 1) was contacted with a cross-linked polydimethylsiloxane (PDMS) membrane with a thickness of 2 μM at room temperature and a transmembrane pressure of 15 bar. The stage cut was 10 weight %, i.e. 10 weight % of the gasoline permeated through the membrane (i.e. retentate was 90 weight % of the inlet stream) and the flux was 150 l/min. The membrane was supported on a support layer of polyacrylonitrile (PAN) with a thickness of 40 μm.

In engine tests, the amount of inlet valve deposits (IVD) and combustion chamber deposits (CCD) were measured for the inlet fuel, the retentate and the permeate by the “Toyota Keep Clean” and “Toyota 1JZ CCD” procedures, respectively, described in EP-B-1230329, at pages 11, 12 and 14. The results are shown in Table 2. The amount of polynuclear aromatics (PNA) in the inlet fuel, the retentate and the permeate was assessed by means of UV absorbance. The results are shown in Table 2.

### Example 2

Example 1 was repeated with a different gasoline inlet stream. The composition and characteristics of the inlet gasoline stream is shown in Table 1. The results are shown in Table 2.

Composition and properties of inlet gasoline

| | Example 1 | Example 2 |
|---|---|---|
| RVP (hPa) | n.a. | 589 |
| Density at 15° C. (kg/litre) | 0.779 | 0.722 |
| RON | 98.8 | 95.2 |
| MON | 86.9 | 87.5 |
| IEP (° C.) | 35.4 | 35.3 |
| FEP (° C.) | 203 | 160.4 |
| E70 | 13.7 | 30.2 |
| E100 | 31.3 | 53.7 |
| paraffins (% v/v) | 10.18 | 5.96 |
| iso-paraffins (% v/v) | 26.94 | 62.87 |
| aromatics (% v/v) | 50.73 | 23.71 |
| sulphur (ppmw) | 138 | 22 |

n.a. not available

TABLE 2
Results

| | Example 1 | | | Example 2 | | |
|---|---|---|---|---|---|---|
| | inlet | | | inlet | | |
| | fuel | retentate | permeate | fuel | retentate | permeate |
| IVD | 260 | 222 | 91 | 30.8 | 25.0 | 7.0 |
| (mg) | | | | | | |
| CCD | 635 | 615 | 631 | 265 | 290 | 257 |
| (mg) | | | | | | |
| PNA | 192.2 | 193.1 | 119.8 | 15.2 | 17.7 | 10.3 |

It can been seen from the results in Table 2 that the quality of the permeate stream is significantly improved as compared to the quality of the inlet stream, especially with respect to cleanliness. The amount of inlet valve deposits and the concentration of polynuclear aromatics has significantly decreased. The quality of the retentate stream has not significantly deteriorated. There is even an improvement in quality with respect to the amount of inlet valve deposits.

1. A process for upgrading a liquid hydrocarbon transportation fuel comprising contacting an inlet stream of liquid hydrocarbon transportation fuel with a non-porous or nano-filtration membrane to produce a first liquid hydrocarbon outlet stream recovered as the retentate and a second liquid hydrocarbon outlet stream recovered as the permeate, wherein the retentate is more than 70 weight % of the inlet stream, and wherein the inlet stream and the first and the second outlet stream each fulfill the requirements for base fuel without further treatment.

2. The process of claim 1 wherein the liquid hydrocarbon transportation fuel is diesel or gasoline base fuel.

3. The process of claim 1 wherein the retentate is at least 80 weight % of the inlet stream.

4. The process of claim 1 wherein the retentate is in the range of from 85 to 95 weight % of the inlet stream.

5. The process of claim 1 wherein the first outlet stream is directly loaded into a transport truck.

6. The process of claim 1 wherein the membrane is a hydrophobic non-porous membrane.

7. The process of claim 6 wherein the membrane is a cross-linked polysiloxane membrane.

8. The process of claim 7 wherein the cross-linked polysiloxane membrane is a cross-linked polydimethylsiloxane membrane or a cross-linked polyoctylmethylsiloxane membrane.

9. The process of claim 6 wherein the membrane is supported on a macroporous or mesoporous support layer.

10. The process of claim 9 wherein the macroporous or mesoporous support layer is a layer of polyacrylonitrile (PAN), polyimide (PI), or polyetherimide (PEI).

11. The process of claim 6 wherein the liquid hydrocarbon transportation fuel is diesel or gasoline base fuel.

12. The process of claim 6 wherein the retentate is at least 80 weight % of the inlet stream.

13. The process of claim 6 wherein the retentate is in the range of from 85 to 95 weight % of the inlet stream.

14. The process of claim 6 wherein the first outlet stream is directly loaded into a transport truck.

15. The process of claim 11 wherein the membrane is a cross-linked polysiloxane membrane.

16. The process of claim 12 wherein the membrane is a cross-linked polysiloxane membrane.

17. The process of claim 13 wherein the membrane is a cross-linked polysiloxane membrane.

18. The process of claim 14 wherein the membrane is a cross-linked polysiloxane membrane.

19. The process of claim 15 wherein the cross-linked polysiloxane membrane is a cross-linked polydimethylsiloxane membrane or a cross-linked polyoctylmethylsiloxane membrane.

20. The process of claim 16 wherein the cross-linked polysiloxane membrane is a cross-linked polydimethylsiloxane membrane or a cross-linked polyoctylmethylsiloxane membrane.

21. The process of claim 17 wherein the cross-linked polysiloxane membrane is a cross-linked polydimethylsiloxane membrane or a cross-linked polyoctylmethylsiloxane membrane.

22. The process of claim 18 wherein the cross-linked polysiloxane membrane is a cross-linked polydimethylsiloxane membrane or a cross-linked polyoctylmethylsiloxane membrane.

23. The process of claim 7 wherein the membrane is supported on a macroporous or mesoporous support layer.

24. The process of claim 11 wherein the membrane is supported on a macroporous or mesoporous support layer.

25. The process of claim 12 wherein the membrane is supported on a macroporous or mesoporous support layer.

26. The process of claim 13 wherein the membrane is supported on a macroporous or mesoporous support layer.

27. The process of claim 14 wherein the membrane is supported on a macroporous or mesoporous support layer.

28. The process of claim 1 wherein the inlet stream contains less than 150 ppmw sulphur.

29. The process of claim 9 wherein the inlet stream contains less than 140 ppmw sulphur.

30. The process of claim 28 wherein the liquid hydrocarbon transportation fuel is diesel or gasoline base fuel.
